(19) United States
(12) Patent Application Publication
(10) Pub. No.: US 2025/0367418 A1
(43) Pub. Date: Dec. 4, 2025

(54) GASTRIC RESIDENCY SYSTEM FOR ELECTRONIC INGESTIBLE DEVICES

(71) Applicant: CELERO SYSTEMS, INC., Lincoln, MA (US)

(72) Inventors: Benjamin David Pless, Lincoln, MA (US); Daniel Bacher, Lincoln, MA (US); Amos G. Cruz, Wrentham, MA (US); Taylor Bensel, East Walpole, MA (US); Nathalie Perry, Methuen, MA (US); Greg Martin, Boston, MA (US)

(21) Appl. No.: 18/854,165

(22) PCT Filed: Apr. 4, 2023
(86) PCT No.: PCT/US2023/065313

§ 371 Date: Oct. 4, 2024

Related U.S. Application Data
(60) Provisional application No. 63/326,935, filed on Apr. 4, 2022.
(60) Provisional application No. 63/327,108, filed on Apr. 4, 2022.

(51) Int. Cl. A61M 31/00; A61B 5/00; A61B 5/07

(52) U.S. Cl. CPC A61M 31/002; A61B 5/073; A61B 5/4839; A61B 5/6861; A61B 2562/162; A61M 2202/0007; A61M 2205/0216; A61M 2205/364; A61M 2210/1053

(57) ABSTRACT

An ingestible device that includes a plurality of retention arms that are configured to retain the ingestible device in a stomach of a subject is provided. The ingestible device includes a sensor to detect a physiological parameter in the stomach of the subject and a controller programmed to process physiological data from the sensor based on the detected physiological parameter.

### CROSS-REFERENCE TO RELATED APPLICATIONS

The present application claims priority to U.S. Provisional Application No. 63/327,108 entitled “Gastric Residency System for Electronic Ingestible Devices” filed on Apr. 4, 2022 and U.S. Provisional Application No. 63/326,935 entitled: “Ingestible Suture Cutting Mechanism” filed on Apr. 4, 2022, both of which are incorporated by reference herein.

### BACKGROUND

Ingestible capsules that incorporate sensors can be useful to monitor the physiological condition of a patient. A capsule format is familiar to patients and, unlike patches or other wearable solutions, compliance is straightforward since the patient simply swallows the capsule at given time periods. However, gastric residency of such ingestible capsules is an important factor to allow for physiological monitoring for an effective period of time.

### BRIEF DESCRIPTION OF THE DRAWINGS

FIG. 1 is a perspective view on an ingestible device in a non-deployed state according to an aspect of the present disclosure.

FIG. 2 is a perspective view of the ingestible device of FIG. 1 in a deployed state.

FIG. 3 is a perspective view of the ingestible device of FIG. 1 with the retention arms released from the ingestible housing.

FIG. 4 is a perspective view of the ingestible device of FIG. 1 illustrating internal components as well as radiopaque markers according to an aspect of the present disclosure.

FIG. 5 is a perspective view of a retention arm of an ingestible device according to an aspect of the present disclosure.

FIG. 6 is a perspective view of a retention arm of an ingestible device according to an aspect of the present disclosure.

FIG. 7 is a perspective view of an ingestible device depicting an embodiment of a retainer of an ingestible device according to an aspect of the present disclosure.

FIG. 8 is a perspective view of an ingestible device depicting an embodiment of a retainer of an ingestible device according to an aspect of the present disclosure.

FIG. 9 is a perspective view of an ingestible device depicting an embodiment of a retainer of an ingestible device according to an aspect of the present disclosure.

FIG. 10 is a block diagram of internal components of an ingestible device according to an aspect of the present disclosure.

FIG. 11 is a block diagram of a controller of an ingestible device according to an aspect of the present disclosure.

FIG. 12 is a side internal schematic view of an ingestible device in a non-deployed state.

FIG. 13 is a side internal schematic view of the ingestible device of FIG. 12 in a deployed state.

FIG. 14 is a side internal schematic view of the ingestible device of FIG. 12 depicting a representative retention arm released from the ingestible housing.

FIG. 15 is a perspective view of an ingestible device according to aspect of the present disclosure.

FIG. 16 is a partial side view of an ingestible device according to an aspect of the present disclosure.

FIG. 17 is a perspective view of an ingestible device according to an aspect of the present disclosure.

FIG. 18 is a schematic illustration of an active retention arm detachment system according to an aspect of the present disclosure.

FIG. 19 is a schematic illustration of an active retention arm detachment system according to an aspect of the present disclosure.

FIG. 20 is a perspective view of an ingestible device according to an aspect of the present disclosure.

FIG. 21 is a cut-away view of the ingestible device of FIG. 20 illustrating a retention arm detachment system according to an aspect of the present disclosure.

FIG. 22 is a close-up view of the retention arm detachment system illustrated in FIG. 21.

FIG. 23 is a schematic view of the retention arm detachment system of FIG. 21 when a representative retention arm is in a non-deployed state.

FIG. 24 is a schematic view of the retention arm detachment system of FIG. 23 when the representative retention arm is in a deployed state.

FIG. 25 is a schematic view of the retention arm detachment system of FIG. 23 illustrating the representative retention arm detached from the ingestible housing of the ingestible device.

FIGS. 26-28 illustrate the sequential steps resulting in detachment of the representative retention arm as depicted in FIG. 25.

FIG. 29 is a schematic view of a retention arm detachment system when a representative retention arm is in a non-deployed state.

FIG. 30 is a schematic view of the retention arm detachment system of FIG. 29 when the representative retention arm is in a deployed state.

### DETAILED DESCRIPTION

The present disclosure relates to an ingestible device comprising retention arms to retain the device in a subject's stomach for an effective period of time. In particular, such retention arms when deployed are too large to fit through the pyloric valve between the subject's stomach and the small intestine. At the end of the device's residency in the stomach, a controller can command the retention arms to release, and the components of the device can be small enough to travel out of the stomach and through the subject's gastrointestinal tract. As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” or “approximately” is meant that the shape, size or configuration of the described element need not have the mathematically exact described shape, size or configuration of the described element but can have a shape, size or configuration that is recognizable by one skilled in the art as generally or approximately having the described shape, size or configuration of the described element. As such “substantially” or “approximately” refers to the complete or nearly complete extent of a characteristic, property, state, or structure. The exact allowable degree of deviation from the characteristic, property, state, or structure will be so as to have the same overall result as if the absolute characteristic, property, state, or structure were obtained. The terms “first,” “second,” etc. are used to distinguish one element from another and not used in a quantitative sense unless indicated otherwise. Thus, a “first” element described below could also be termed a “second” element. A component that is “connected to,” “coupled to,” “in communication with” “linked to” or “joins” another component can have intervening components between the components so long as the device can perform the stated purpose. The terms “top,” “bottom,” “down,” “downward,” “up,” “upward,” “inner,” “proximal,” “distal,” and “laterally” refer to the position or location of components as they are depicted in the drawings. An “effective period of time” is the residency duration (the time the ingestible device is retained in the stomach) necessary for the device to perform its disclosed function. As used herein, a “subject” includes a mammal such as a human being.

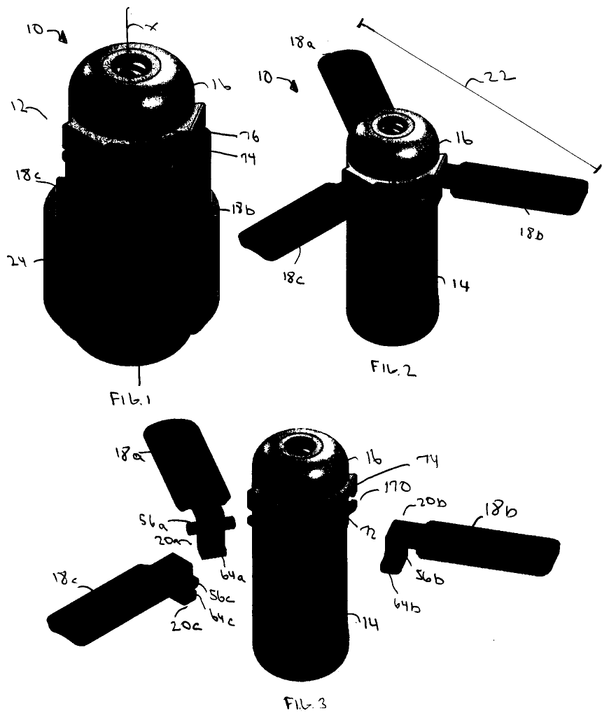

Referring to FIGS. 1-4, in an aspect, an ingestible device 10 can comprise ingestible housing 12 including housing body 14 having a longitudinal axis X and cap 16 displaceably coupled to a top portion of housing body 14. The ingestible device can take the form of a capsule (e.g. a 000 sized capsule) or tablet, for example. The capsule or tablet can be clear or opaque. The device can further include a coating (e.g. a coating including a gelatin coating) or otherwise be encapsulated by a dissolvable material. The cap and housing body can be displaceably coupled via fastener 68. For example, the fastener can be a screw, bolt, pin, a male/female fastener or the cap and housing body can be displaceably coupled via an interference fit. A plurality of retention arms 18 each having an inner portion 20 can be releasably connected to housing body 14, cap 16 or both. Although the figures illustrate three retention arms, the device can include more or less than three arms so long as the arms retain the device in the stomach for an effective period of time. Referring to FIG. 1, in a non-deployed state, plurality of retention arms 18 are folded down along the longitudinal axis X of housing body 14. Referring to FIGS. 2 and 4, in a deployed state, plurality of retention arms 18 extend laterally about the housing body. The plurality of retention arms can be sized and configured to retain the ingestible housing in the stomach of a subject in a deployed state. For example, each of the retention arms can have a “wingspan” 22 (tip to tip distance between adjacent retention arms) such that the device is too large to pass through the subject's stomach pylorus, thereby temporarily retaining the device in the subject's stomach for an effective period of time. For example, the wingspan can be approximately 3.5 centimeters (cm) to approximately 4.5 cm.

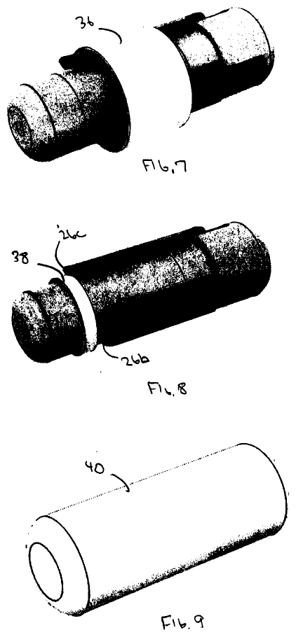

Referring to FIG. 5, in certain aspects, retention arm 18A can include proximal segment 24 comprising a rigid material or semi-rigid material (e.g. thermoplastic material such as polycarbonate plastic or polyetheretherketone (PEEK)) and distal tip segment 26 can comprise a soft, flexible material (e.g. silicone or a flexible thermoplastic material such as, for example, thermoplastic polyurethane (TPU)). Referring to FIG. 6, in certain aspects retention arm 18B can include a proximal segment 28 comprising a rigid or semi-rigid material (such as described with respect to FIG. 5) and distal segment 30 can comprise a softer, more flexible material where distal segment 30 can comprise a protrusion 32 of proximal segment 28 overmolded with covering 34 comprising a flexible material (e.g. a flexible thermoplastic material such as, for example, thermoplastic polyurethane (TPU)). The soft distal segment can minimize possible tissue irritation or damage when the tips of the retention arms press against tissue in the stomach or other part of the gastrointestinal tract.

Referring to FIG. 4, in certain aspects, the retention arms and/or the housing body can include radiopaque markers 70 such the components of the device can be identified and tracked via X-ray. Although FIG. 4 illustrates radiopaque markers on only one retention arm, radiopaque markers could be included in other retention arms as well.

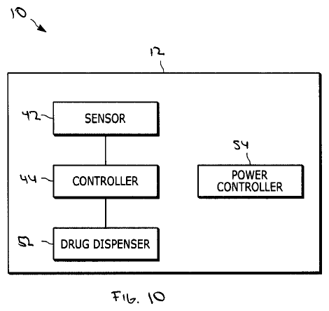

Ingestible device 10 can further include a dissolvable retainer, sleeve or retainer 24 disposed about the ingestible housing configured to retain the plurality of retention arms in a non-deployed state until dissolved wherein the plurality of retention arms assume a deployed state as illustrated in FIGS. 2 and 4. Referring to FIG. 7, the retainer can be a band 36 disposed about the entire outer surface of ingestible housing. Alternatively, the retainer can be a band 38 adhered to tips 26 of the plurality of retention arms as illustrated in FIG. 8 via an adhesive. A non-limiting example of an adhesive is cyanoacrylate. Still alternatively, the can be shell 40 that fully encapsulates the ingestible housing as illustrated in FIG. 9. The retainer can be a continuous structure or a discontinuous structure. For example, the retainer can be separate pieces (e.g. three separate pieces) where each piece holds down a respective one of the plurality of retention arms. The retainer can comprise a water soluble material such as, for example, polyvinyl alcohol (PVA) or gelatin. In all configurations, after the ingestible device is swallowed, the retainer can dissolve in a few minutes and the elastic or elastomeric member inside the housing body (described below) can bias the cap downward towards the housing body resulting in the retention arms rotating laterally to assume a deployed state.

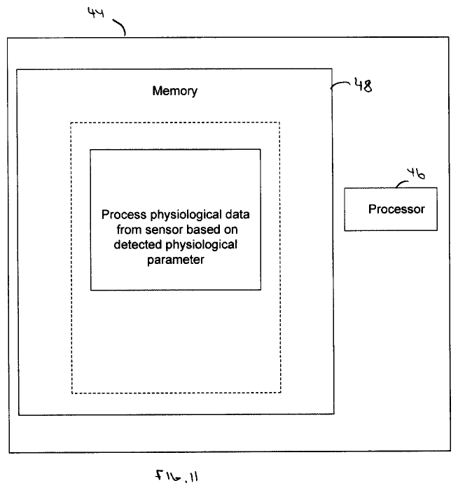

Referring to FIGS. 10-11, ingestible device 10 can further comprise sensor 42 contained within ingestible housing 12 configured to detect a physiological parameter in the stomach. Non-limiting examples of sensors include an accelerometer, an electrocardiogram (ECG) sensor, a photoplethysograph (PPG) sensor, a temperature sensor, or combinations thereof. Non-limiting examples of physiological parameters include electrical cardiac activity, heart rate, heart rate variability, respiratory monitoring (e.g. respiratory rate), saturated oxygen, intestinal tissue color, central temperature, bodily motions as detected from the stomach, or combinations thereof. Controller 44 can also be contained within the ingestible housing and can comprise processor 46 and memory 48. Memory 48 can include computer-executable instructions 50 stored thereon, that when executed by processor 46 cause controller 44 to process physiological data from sensor 42 based on the detected physiological parameter. The ingestible device can further include a drug dispenser 52 containing a therapeutic agent. Examples of an ingestible device with drug dispensers are described in U.S. Provisional Application No. 63/451,718 entitled: “Ingestible Electronically Triggered Drug Delivery System” filed on Mar. 12, 2023 and incorporated by reference herein. Exemplary therapeutic agents include an opioid antidote including opioid antagonists such as, for example, nalmefene, naltrexone, samidorphan, buprenorphine, or suitable combinations thereof. The memory can further store computer-executable instructions that, when executed by the processor, actuate release of the therapeutic agent from the drug dispenser into the subject upon a determination that the physiological parameter falls outside a threshold value or range for the physiological parameter. Description of release of a therapeutic agent from a drug dispenser upon a determination that a physiological parameter falls outside a threshold value or range for the physiological parameter are described in U.S. application Ser. No. 17,007,086 (“the '086 application”) entitled: “Opioid Overdose Rescue Device” filed on Aug. 31, 2020 and incorporated by reference herein. The ingestible device can further comprise a power controller 54, other sensors, a radio, antenna and other components as described in the '086 application, such disclosure incorporated by reference herein.

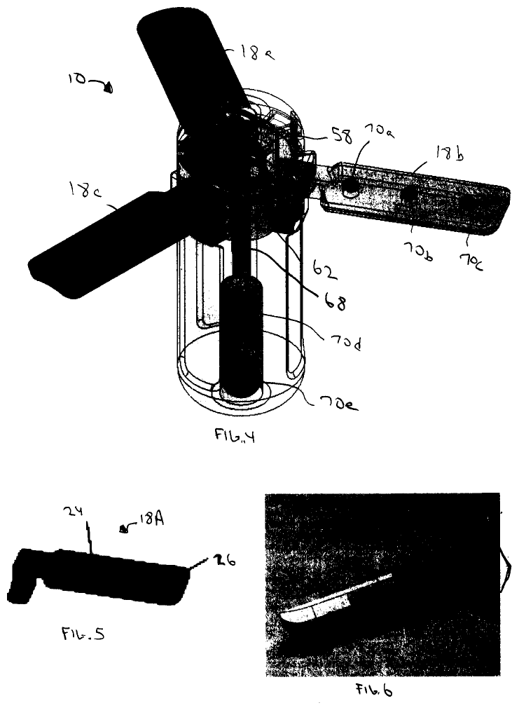

With reference FIGS. 12-14 (which depict one representative retention arm for sake of clarity) in conjunction with FIGS. 3 and 4, ingestion device 10 can further comprise a plurality of pins 56, each of the plurality of pins extending through the inner portion 20 of a respective one of the plurality of retention arms 18. Each of the plurality of retention arms 18 can be rotatable about a respective one of the plurality of pins 56. Inner portions 20 of retention arms 18 can be disposed in socket 66 defined by housing body 14 and cap 16. The ingestible device can further comprise an elastic or elastomeric member 58 having one end in communication with interior top surface 60 of cap 16 and another end in communication with flange 62. The elastic or elastomeric member can be any member that stores mechanical energy and releases it when the opposing force is removed. For example, the elastic or elastomeric member can be a spring (e.g. a metal spring). The elastic or elastomeric member can be configured to bias flange 62 towards each of the inner portions 20 of the plurality of retention arms 18 when the dissolvable retainer dissolves thereby initiating outward lateral rotation of each of the plurality of retentions arms 18 about a respective one of the plurality of pins 56. Inner portion 20 of each of the plurality of retention arms 18 can also comprise lever 64. The elastic or elastomeric member can be configured to bias flange 62 towards lever 64 of each of the plurality of retention arms 18 to latch the plurality of arms in a “locked” position.

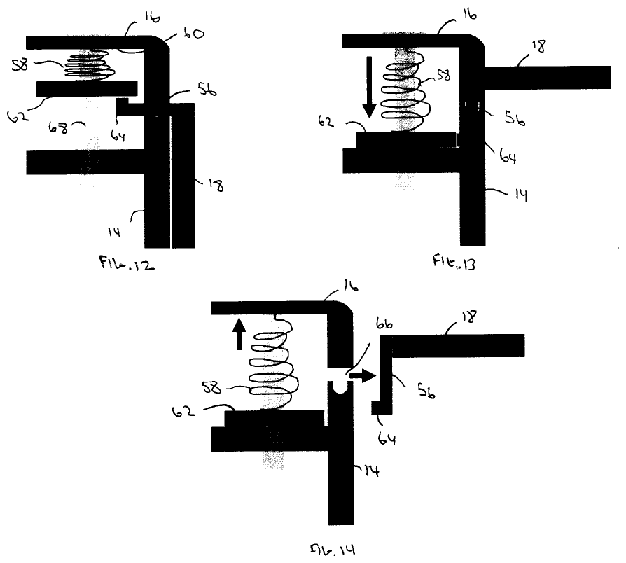

FIG. 12 illustrates the internal components of ingestible device 10 is a non-deployed state. After the device is swallowed by a subject and reaches the stomach, the dissolvable retainer can dissolve thereby allowing potential energy stored in elastic or elastomeric member 58 (which is illustrated in FIG. 12-14 as a compression spring but could be another structure) to bias flange 62 downward towards inner portion 20 of each retention arm 18 as illustrated in FIG. 13. When flange 62 contacts inner portion 20, each retention arm 18 rotates laterally outwards about pin 56 until each retention arm achieves a desired angle (for example 90 degrees or another specified angle) between the retention arm and the housing body as illustrated in FIG. 13. Flange 62 is displaced a sufficient distance to latch lever 64 of each retention arm 18 such that each retention arm is “locked” in the outward deployed state. The force required to break the retention arms free from the socket is higher than the gastric forces generated in the stomach so that the ingestible device is retained in the stomach. After an effective period of time (e.g. the device's use life), the retention arms can detach from the ingestible housing as described in more detail below.

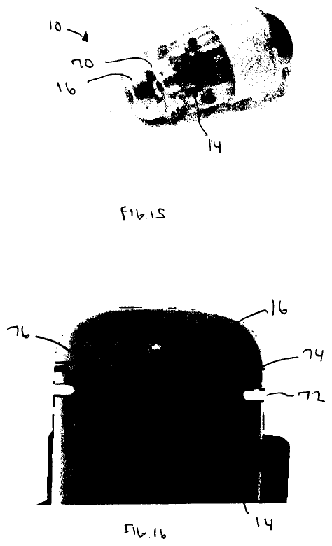

For example, and with reference to FIGS. 15 and 16 in conjunction with FIG. 1, ingestible capsule 10 can include releasable filament 70 that joins cap 16 and housing body 14 whereby remove of the releasable filament causes cap 16 to bias away from housing body 14 allowing detachment of plurality of retention arms 18. The filament can include, for example, a monofilament, multifilament, a thread, wire, suture, or a filament comprising multiple materials and/or having dimensions that vary along its length. Referring to FIG. 16, channel 72 can be located between the bottom of cap 16 and the top of housing body 14. Housing body 14 can include filament channel 74 that is substantially aligned with filament channel 76 of cap 16 (such filament channels also illustrated in FIG. 1). Filament 70 can be disposed in channels 72, 74 and 76. As shown in FIG. 15, the filament can the tied with a slip knot, for example, to allow the filament to be pulled free from the via an endoscopic grasper, for example, during an endoscopic procedure when the device is in the subject's stomach. When the releasable filament is pulled free, the elastic or elastomeric member can force the cap to lift away from the housing body a sufficient distance for the pins extending through the retention arms to slide out of the respective sockets, thereby releasing the retention arms from the housing body as illustrated in FIGS. 3 and 14. The offset between the housing body filament channel and the cap filament channel can be seen FIG. 3. When the retention arms detach, the ingestible housing and the retention arms are now small enough to pass through the subject's pylorus to travel through the rest of the gastrointestinal tract to be excreted naturally.

In certain aspects, the releasable filament can comprise an absorbable material such that over time, the filament can degrade and weaken sufficiently for the elastic or elastomeric material to separate the housing body and cap, allowing the retentions arms to detach from the ingestible housing and allowing the ingestible device components to pass out of the stomach. This allows the residency duration (the time the device is retained in the stomach) to be controlled by the dissolution time of the absorbable/degradable releasable filament material.

Alternatively or in addition to passive degradation of the releasable filament, the releasable filament can be actively detached. For example, the releasable filament can be severed or melted. Regarding the latter and with reference to FIG. 17, the ingestible housing can comprise microcontroller 78 electrically connected to thermal contact 80 that is in communication with releasable filament 70. Microcontroller 78 can be programmed to deliver electrical current to thermal contact 80 to melt or weaken the releasable filament. In particular, the releasable filament can be melted using a thermal contact controlled by a microcontroller. Electrical current from the microcontroller can be delivered via a conductor 82 to thermal contact 80. The heat from the thermal contact can reach a temperature above the melting point of the releasable filament causing the releasable filament to weaken thereby losing tensile strength to allow the elastic or elastomeric member to separate the cap from the housing body thereby releasing the retention arms. The thermal contact can comprise tungsten, for example, that heats up to a temperature above 100° C. when electrical current flows through the thermal contact. The releasable filament can comprise a material that has a lower melting point such as, for example, polycaprolactone, that can melt at a lower temperature such as, for example, 60° C. to 80° C.

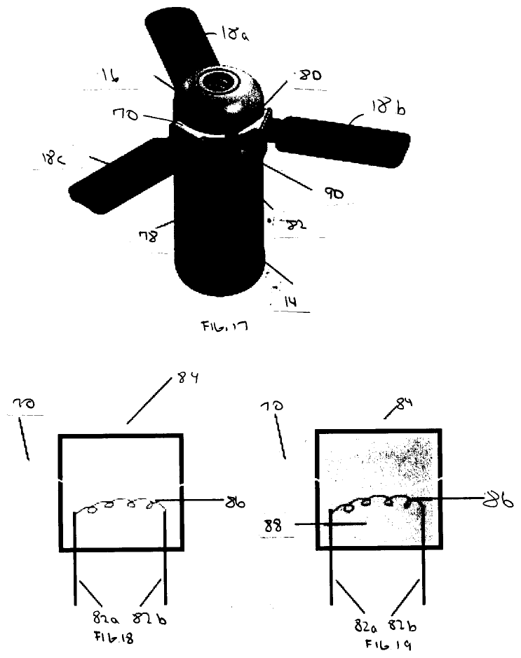

Referring to FIGS. 18 and 19, the ingestible housing can comprise chamber 84 comprising heating filament 86 (e.g. tungsten) electronically connected to a microcontroller (as described above). Electrical current from the microcontroller can be delivered via conductors 82a and 82b to heating filament 86. Releasable filament 70 can extend through chamber 84 and be in communication with heating filament 86. Referring to FIG. 19, chamber 84 can additionally comprise an additional chemical reactant that causes an exothermic reaction to generate additional heat beyond the heat energy provided by electrical current flowing through heating filament 86. Such an embodiment can provide additional heat to ensure releasable filament 70 melts sufficiently to detach from the ingestible housing to allow release of the retention arms. Additional or alternative details of a related releasable filament cutting chamber are disclosed in U.S. Provisional Application No. 63/326,935 (“the '935 application”) entitled: “Ingestible Suture Cutting Mechanism” filed on Apr. 4, 2022 and incorporated by reference herein. The chemical reactant can comprise a combustible material, a thermochemical reactant, a propellant or another reactant that causes an exothermic reaction. Non-limiting examples, of a combustible material include a mixture of carbon, sulfur, potassium nitrate or similar chemicals that will ignite when a sufficient temperature is reached from the heating filament leading to an exothermic chemical reaction that will rapidly melt and cut the releasable filament. Other exemplary chemical reactants and their mechanisms of action are disclosed in the '935 application, which is incorporated by reference herein.

Another example of an active retention arm detachment device is a chamber similar to chamber 84 through which releasable filament 70 passes and that, when electrically triggered, severs the releasable filament with a sharp edge as disclosed in the '935 application, which is incorporated by reference herein.

In an aspect, conducting contacts 90 can be disposed at the location where housing body 14 and cap 16 interface. When releasable filament 70 is intact and cap 16 is in contact with the housing body 14, the conducting contacts 90 would be closing a circuit that can be connected to microcontroller 78 to be used to detect if the assembly is intact and retention arms 18 are attached the ingestible housing. When the releasable filament is detached and the cap and the retention arms are released, the conducting contacts 90 would separate, resulting in an open circuit, which could be measured by microcontroller 79, thereby allowing the microcontroller to detect when the retention arms have detached. Such an embodiment can provide closed loop feedback to the thermal contact and/or heating filament to control the device to ensure proper release of the retention arms.

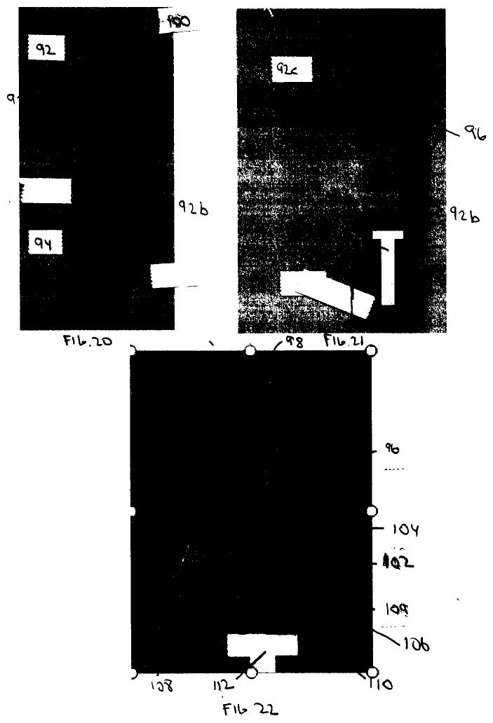

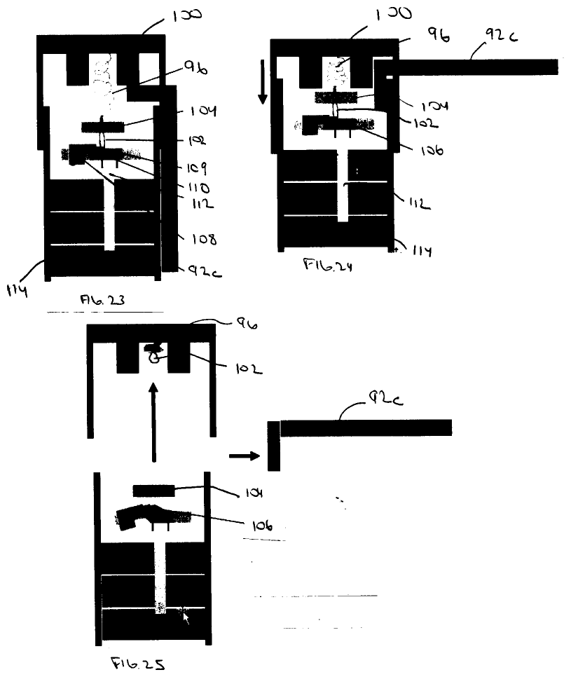

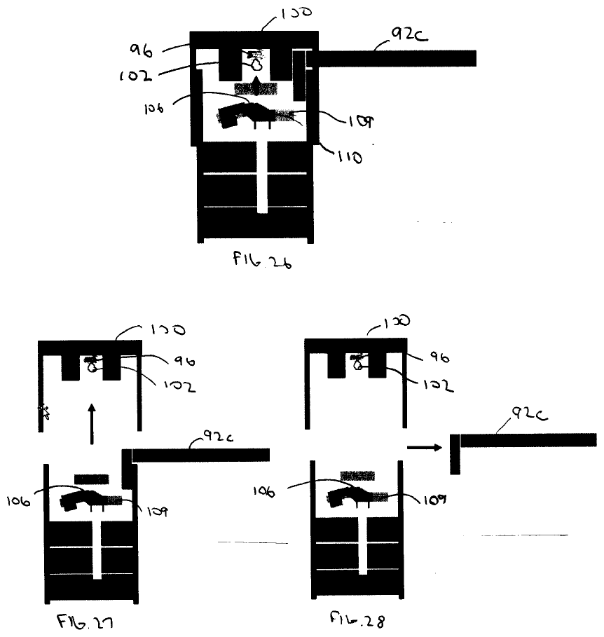

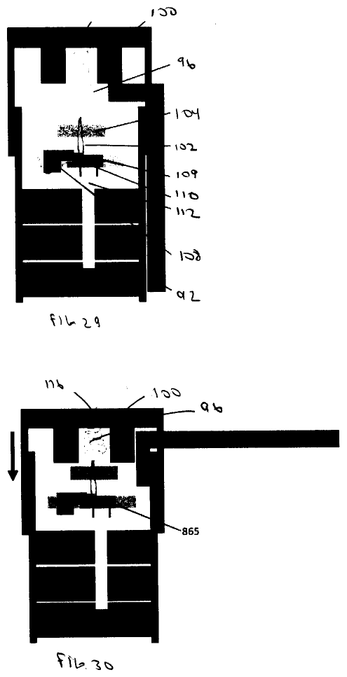

An ingestible device as disclosed herein can include other retention arm detachment systems. The device can comprise an elastic or elastomeric member configured to bias the cap towards the housing body and can have one end in communication with an inner top surface of the cap and another end in communication with a deformable member contained within the housing body. The deformable member can have a heating filament or other type of heating contact embedded therein or otherwise in communication therewith such that when the deformable member is heated, the force from the elastic or elastomeric member can deform the deformable member allowing the elastic or elastomeric member to release from the deformable member. FIGS. 20-28 illustrate a retention arm detachment system that includes such components. FIG. 20 illustrates ingestible device 92 with one representative retention arm 92c in a deployed state and another representative retention arm 92b in a non-deployed state for ease of visualization of both device states. FIG. 21 shows ingestible device 92 with housing body 94 removed to visualize the components of the retention arm detachment system. FIG. 22 is a close-up view of the internal components of the retention arm detachment system illustrated in FIG. 21. The system can include an elastic or elastomeric member 96 having one end in communication with an inner top surface 98 of cap 100 and another end in communication (e.g. mechanically linked) with looping filament 102 (which can comprise, for example, a wire, a thread, monofilament, multifilament, suture etc.) In certain aspects the elastic or elastomeric member is an extension spring (e.g. a metal spring). Elastic or elastomeric member 96 is configured to bias cap 100 towards housing body 94 (e.g. apply a force to pull cap 100 toward the housing body 94) that deploys the retention arms 92. Looping filament 102 can pass through septum 104, which can be fabricated of silicone, for example, and can loop onto a rotation pin 106 of rotation collar 108. In certain aspects, the system does not include a rotation collar. Rotation pin 106 can be mechanically coupled to a low-melting point thermoplastic pin 109, which can have a heating filament or contact 110 embedded inside of it. Heating filament or contact 110 can be electrically connected to flexible circuit connector 112, which can connect to device circuit board 114. In its default state, thermoplastic pin 109 can retain rotation pin 106/collar 108 in a fixed position and angle, despite the force elastic or elastomeric member 96 applies via looping filament 102, thereby holding cap 100 and housing body 94 together. To actively detach elastic or elastomeric member 96, device circuit board 114 can deliver electrical current through heating filament or contact 110. When sufficient heat is generated above the melting point of thermoplastic pin 109, pin 109 can soften, allowing rotation pin 106/collar 108 to rotate (e.g. counterclockwise as depicted in FIG. 26) resulting in looping filament 102 sliding off rotation pin 106 as illustrated in FIG. 26. Elastic or elastomeric member 96 can then be allowed to further compress and apply tension to looping filament 102 to pull looping filament 102 upwards through septum 104, and therefore completely release the cap 100 from housing body 94 as illustrated in FIG. 27. As is shown in FIG. 28, when cap 100 is detached from housing body 94, cap 100 can lift off housing body 94, and retention arms 92 can be free to detach from cap 100 and housing body 94. In particular, once the elastic or elastomeric member is no longer retaining the cap on the housing body. the parts can be free to separate from each other due to motion within the stomach. As the cap detaches, the arms can then also be free to detach due to motion in the stomach. As stated above, the elastic or elastomeric member may be an extension spring comprising stainless steel, for example, or in an alternative embodiment could be replaced with a rubber band with similar force characteristics. The low melting point thermoplastic pin can comprise, for example, a polymer such as polycaprolactone (PCL), which has known biocompatible properties and melting points of approximately 80° C. The heating filament or contact can comprise tungsten or other metal coiled filament, for example, that generates heat sufficient to melt the thermoplastic pin when supplied with current from the circuit board.

In certain aspect, the looping filament can be replaced with another structure that couples the elastic or elastomeric member to the heating filament or contact. Such a structure can be a rigid or semi-rigid plastic member, a filament with a knot or ball at the end, etc. in communication with the heating filament or contact.

Referring to FIGS. 29-30, in certain aspects, degradable pin 116 disposed in cap 100 and coupled to elastic or elastomeric member 96 can retain cap 100 onto housing body 94. Thus, if the rotation pin does not rotate for some reason to allow the looping filament to slide off the rotation pin, degradable pin 116 there would be a “backup” component that can eventually degrade and detach the cap and retention arms.

As stated above, the retention arms allow the device to remain in the subject's stomach for an effective period of time. For example, in the case where the ingestible device includes a sensor that detects a physiological parameter, an effective period of time can be the time period it takes to detect the physiological parameter such that the physiological parameter can be processed by the controller or transmitted to an external device for analysis. In the case where the ingestible device also includes a therapeutic agent, an effective period of time can be the time it takes to release the therapeutic agent into the stomach. In certain aspects, the effective period of time for residency duration of the device in the stomach is approximately one week. Each of the separate components of the ingestible device can be small enough to pass through the pylorus of the subject after an effective period of time and safely pass through the gastrointestinal tract for excretion.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. An ingestible device comprising:
an ingestible housing comprising a housing body having a longitudinally axis and a cap displaceably coupled to a top portion of the housing body;
a plurality of retention arms each having an inner portion releasably connected to the housing body, the cap, or both and having a non-deployed state wherein the plurality of retention arms are folded down along the longitudinal axis of the housing body and a deployed state wherein the plurality of retention arms extend laterally about the housing body, the plurality of retention arms sized and configured to retain the ingestible housing in a stomach of a subject in a deployed state;
a dissolvable retainer disposed about the ingestible housing configured to retain the plurality of retention arms in a non-deployed state until dissolved wherein the plurality of retention arms assumes a deployed state;
a sensor contained within the ingestible housing configured to detect a physiological parameter in the stomach of the subject; and
a controller contained within the ingestible housing comprising a processor and memory, the memory having computer-executable instructions stored thereon that, when executed by the processor, cause the controller to process physiological data from the sensor based on the detected physiological parameter.

2. The device of claim 1, further comprising a drug dispenser contained within the ingestible housing and comprising a therapeutic agent.

3. The device of claim 2, further comprising computer-executable instructions stored in the memory that, when executed by the processor, actuate release of the therapeutic agent from the drug dispenser into the subject upon a determination that the physiological parameter falls outside a threshold value or range for the physiological parameter.

4. The device of claim 1, further comprising a plurality of pins, each of the plurality of the plurality of pins extending through the inner portion of a respective one of the plurality of retention arms, each of the plurality of retention arms rotatable about a respective one of the plurality of pins.

5. The device of claim 4, further comprising an elastic or elastomeric member having one end in communication with an interior top surface of the cap and another end in communication with a flange, the elastic or elastomeric member configured to bias the flange towards each of the inner portions of the plurality of retention arms when the dissolvable retainer dissolves thereby initiating outward lateral rotation of each of the plurality of retention arms about a respective one of the plurality of pins.

6. The device of claim 4, wherein the elastic or elastomeric member is a compression spring.

7. The device of claim 1, further comprising an extension spring having one end in communication with an interior top surface of the cap and configured to bias the cap away from the housing body when the dissolvable retainer dissolves thereby initiating outward lateral rotation of each of the plurality of retention arms.

8. The device of claim 5, wherein the inner portion of each of the plurality of retention arms comprises a lever, the elastic or elastomeric member configured to bias the flange towards the lever of each of the plurality of retention arms to latch the plurality of retention arms in a locked position.

9. The device of claim 1, further comprising a filament disposed about the cap and the top portion of the housing body to displaceably couple the cap to the top portion of the housing body wherein removal of the releasable filament causes the cap to bias away from the housing body allowing detachment of the plurality of retention arms.

10. The device of claim 9, wherein the releasable filament comprises an absorbable or degradable material.

11. The device of claim 9, wherein the ingestible housing further comprises a microcontroller electronically connected to a thermal contact that is in communication with the releasable filament, the microcontroller programmed to deliver electrical current to the thermal contact to melt or weaken the releasable filament.

12. The device of claim 9, wherein the ingestible housing further comprises:
a chamber through which the releasable filament extends;
a heating filament contained within the chamber and in communication with the releasable filament;
a microcontroller electronically connected to the heating filament programmed to deliver electrical current to the heating filament; and

13. The device of claim 12, further comprising a chemical reactant that causes an exothermic reaction to generate additional heat beyond the heat energy provided by the electric current flowing through the heating filament to melt or weaken the releasable filament.

14. The device of claim 1, further comprising:
a plurality of conducting contacts located at the interface of the ingestible housing body and the cap; and
a microcontroller electronically connected to the conducting contacts and programmed to determine whether the retention arms are connected to the housing body.

15. The device of claim 1, wherein each of the plurality of retention arms comprises a proximal segment comprising a rigid or semi-rigid material and a distal tip comprising a flexible material.

16. The device of claim 1, further comprising:
a rotation pin contained within the housing body;
a thermoplastic pin in communication with the rotation pin and configured to retain the rotation pin in a fixed position and angle;
a heating filament embedded in the thermoplastic pin;
a looping filament looped about the rotation pin; and
an elastic or elastomeric member having one end in communication with an interior top surface of the cap and another end in communication with the looping filament, the elastic or elastomeric member configured to bias the cap towards the housing body.

17. The device of claim 16, wherein the rotation pin extends from a rotation collar contained within the housing body.

18. The device of claim 16, further comprising a degradable pin disposed in the cap and coupled to the elastic or elastomeric member configured to temporarily retain the cap onto the housing body.
